(19) United States
(12) Patent Application Publication
(10) Pub. No.: US 2017/0319066 A1
(43) Pub. Date: Nov. 9, 2017

(54) METHOD AND APPARATUS FOR MEASURING BODY CORE TEMPERATURE AND CORE TO SKIN TEMPERATURE GRADIENTS

(71) Applicant: Morelight Technologies, Inc., Grosse Pointe, MI (US)

(72) Inventors: Benjamin Ver Steeg, Redlands, CA (US); Craig William White, Grosse Pointe, MI (US)

(21) Appl. No.: 15/587,387

(22) Filed: May 4, 2017

Related U.S. Application Data
(60) Provisional application No. 62/332,125, filed on May 5, 2016.

(51) Int. Cl. A61B 5/00; A61B 5/01; G01K 5/22; A61M 39/02

(52) U.S. Cl. CPC A61B 5/0008; G01K 5/22; A61B 5/7246; A61B 5/01; A61B 5/443; A61B 5/4875; A61B 5/7257; A61B 5/725; A61B 5/0075; A61B 5/683; A61B 5/4869; A61B 2560/0223; A61B 2562/0233; A61M 39/0247

(57) ABSTRACT

A temperature measurement device, comprising a spectral measurement system, configured to determine the absorbance of tissue at a plurality of frequencies, and an analysis system, configured to determine the temperature of the tissue from the determined absorbance, wherein water has a temperature dependent absorption at one of the plurality of frequencies that is different than that at another of the plurality of frequencies.

### TECHNICAL FIELD

The present disclosure relates to a quantitative spectroscopy system for measuring the body core temperature in biological tissue and/or humans utilizing non-invasive techniques in combination with multivariate analysis. Specifically, this method provides depth resolved temperature information such that the temperature of the body can be determined by superficially (skin temperature) and within deep structures (core temperature) and at varying locations as desired.

### BACKGROUND

Significant research and development has been directed at methods to improve and enhance performance of professional and amateur athletes in various sports and activities. In addition, the military is interested in monitoring and improving the sustained performance of personnel in combat situations.

Climatic injuries, including hypothermia, hyperthermia and heat stroke, are common in many sports activities, in addition to muscle cramps and heat stroke.

Real time temperature monitoring is also of great importance is in the health management of the elderly. Up to 20 percent of the elder population is incapable of sweating and are therefore susceptible to heat related injuries. Others often suffer from extremely low core body temperatures.

Additional recognized applications for wearable core temperature monitors include but are not limited to: fertility tracking via basal temperature fluctuations, early detection of viral or bacterial infections, critical care patient monitoring, and estimation of metabolic activity in response to diet or exercise. Additionally, the combination of optical core temperature monitoring with other optical measurements such as tissue oxygenation, can be used to provide more accurate health information.

Numerous publications and papers have identified the monitoring of body core temperature as a key indicator that can influence individual performance and can help to prevent injuries. In addition, recent literature suggests that the difference between skin and core temperature can be a leading indicator of potential heat related problems and injuries.

While a variety of skin temperature sensors, both wearable and laboratory based, are available in the market, a limited set of continuous core temperature sensors are available which are suitable for routine use, especially during exercise. Examples of these include:
Basic mercury thermometer—this has been used for a long time to monitor if you have a fever. It is held in the mouth to measure oral temperature, and sometimes under the armpit for a period of time. Neither method is suitable for use for during exercise.
Ear thermometer—these are becoming popular as a method for measuring deep body (core) temperature and they also rely on optical detection methods. These sensors rely on directly imaging the tympanic membrane temperature which is susceptible to time lags and local infections. Additionally, these systems are not able to provide depth resolved temperature information.
Rectal temperature—the use of a rectal probe in the past has been a common method to measure body temperature during exercise testing, and is one of the most accurate methods available but is not suitable for continuous measurements.
Esophageal temperature—this method is not popular because of the difficulty of inserting the thermistor, irritation to the nasal passages and general subject discomfort during monitoring.
Gastrointestinal radio pill—a small pill is swallowed which gives off a radio signal which is picked up by a monitor outside the body. Each pill is for a single use of course, and is quite expensive.

In order to optimize training protocols, or aid in the heat management for the elderly, and potentially head off heat related injury through predictive means, a fast, accurate, reliable, and non-invasive method for measuring body core temperature is required. In addition, to obtain the most flexibility of response and gauge the influence of a variety of other factors, a method of measuring both core temperature and skin temperature in a single device is desired.

### SUMMARY OF THE INVENTION

The present teachings generally relate to the determination of body core temperature through measurement of certain water or hydration characteristics by a non-invasive spectroscopic system.

Quantitative spectroscopy systems suitable for measuring the presence or concentration of water, hydration levels, hydration state, lactose, lactate, collagen, proteins, or a combination thereof utilizing non-invasive techniques in combination with multivariate analysis has been disclosed in application Ser. No. 14/164,782, filed Jan. 27, 2014, which is incorporated herein by reference.

Such systems overcome the challenges posed by the spectral characteristics of biological samples by incorporating a design that includes, in some embodiments, six optimized subsystems. The design contends with the complexities of the tissue spectrum, high signal-to-noise ratio and photometric accuracy requirements, tissue sampling errors, calibration maintenance problems, calibration transfer problems plus a host of other issues. The six subsystems include an illumination subsystem, a tissue sampling subsystem, a spectrometer subsystem, a data acquisition subsystem, a computing subsystem, and a calibration subsystem. These subsystems can be combined in a single physical implementation (e.g. an LED can serve as both an illumination and a spectrometer subsystem in certain implementations).

A method described here for determining body core temperature makes use of the well-defined spectral absorbance properties of water. Spectral absorbance profiles of water change as a function of temperature. A profile of absorbance versus frequency for water is available in the literature. An examination of the profile shows that the absorbance characteristics of water is significantly greater at certain frequencies than at others. The measurement of absorbance at key frequencies within the spectrum of interest where water is known to change absorbance with changes of temperature is compared to other key frequencies where the change is minimal.

A simple ratio of the absorbance level in the temperature sensitive frequency range to the absorbance level in the temperature insensitive frequency range yields correlation to body core temperature to within 10%. To achieve greater accuracy one must compensate for actual water content and volume, other absorbers in the tissue, the scattering effect of light in tissue as skin compresses due to pressure or change in water content, among others.

For the purpose of core temperature measurement, only a small subset of frequencies within the range defined for body hydration are necessary although more frequencies improve accuracy. Thus, a system designed to measure body hydration can also measure core temperature through the present method.

The depth of measurement can be set as a function of frequency and spacing of the tissue interrogation system (e.g., light source and light collector spacing). For core temperature correlation, measurement can be taken from a deeper part of the tissue. For temperatures more closely correlated with skin temperatures, a shallower depth is desired. In addition to the use of variable source—receiver separations, other suitable methods of achieving depth resolved temperature information include: wavelength specific depth profiles based on water absorbance, time-frequency modulation of light sources using time-of-flight or phase sensitive detection methods, and optical polarization sensitive detection methods.

A system comprised of two different light source—collector spacings can be used to measure core/skin temperature differentials.

A system using multiple, wavelength selective light sources can be used in place of separate illumination and spectrometer subsystems. Example light sources include LEDs and laser diodes. The light source elements can be modulated to provide synchronous detection methods for wavelength information encoding and noise reduction.

A system using multiple, wavelength selective detector elements can be used in combination with one or more broad band light sources to provide a spectrometer subsystem. Example embodiments include the use of single LEDs in combination with multiple absorbance filters to provide multiple wavelength-specific channels of information from a single LED. The use of blackbody filament light sources in combination with multiple filter elements can also be a suitable embodiment.

For combination measurements where the optical detection of core temperature is combined with other diagnostic tests that require a more extensive spectrometer subsystem, a full spectrometer subsystem can provide the required information for the core temperature measurement as well as the other diagnostic tests.

Temperature dependent water absorption features span several different wavelength regions. Suitable options for temperature determination have been demonstrated in both the Silicon detection regime (less than 1,100 nm) as well as in the InGaAs detection regime. Embodiments have been demonstrated in which the selected wavelengths are chosen in order to optimize the instrumentation design (size, cost, etc.) around a specific detector architecture.

Another example embodiment can use a commercially available skin temperature sensor, such as a calibrated thermistor in conjunction with the spectral based core temperature.

Further areas of applicability will become apparent from the description provided herein. The description and specific examples in this summary are intended for purposes of illustration only and are not intended to limit the scope of the present disclosure

### DRAWINGS

The drawings described herein are for illustrative purposes only of selected example embodiments, and are not intended to limit the scope of the present disclosure.

FIG. 1 is an illustration of results from a recent study showing the correlation between core body temperature determined according to the present invention and core body temperature determined from a Gastrointestinal radio pill.

FIG. 2 is a schematic depiction of a non-invasive analyte measurement system suitable for use in the present invention.

FIG. 3 is a schematic depiction of a non-invasive analyte measurement system suitable for use in the present invention.

FIG. 4 is a schematic depiction of a non-invasive hydration measurement device system where the illumination subsystem and spectrometer subsystems are implemented as a combined illumination/modulation subsystem

FIG. 5 is a drawing showing the temperature dependence of water spectral absorption as a function of temperature.

FIG. 6 shows the general profile of a broad spectrum analyte measurement system with example specific frequencies identified for discrete measurement of temperature.

FIG. 7 is a schematic of an example system for core body temperature measurement.

FIG. 8 is a schematic of an example system for core and skin temperature measurements by spectral methods in accord with the present invention.

FIG. 9 is an illustration of the correlation of a simple 1 or 2 frequency system to determinations using a swallowed Gastrointestinal radio pill.

FIG. 10 is a schematic representation of an example data acquisition subsystem.

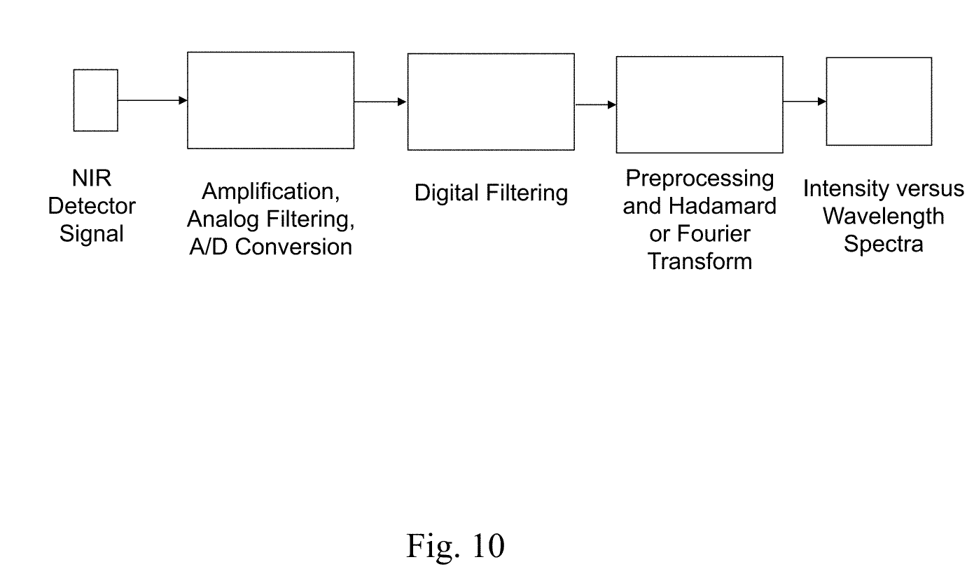

FIG. 11 is a schematic depiction of a sampler providing multiple spacing for emitter/receiver separation.

FIG. 12 is a graphical representation illustrating the benefits of a two-channel sampling subsystem.

FIG. 13 is a schematic representation of representative tissue path lengths from multiple spacings.

FIG. 14 shows the effective path length versus wavenumber for a sampling subsystem of a noninvasive hydration/core temperature sensor used in a human hydration application.

FIG. 15 is a diagram of an integrated sampling subsystem suitable for use in the present invention.

FIG. 16 is a schematic representation of an example embodiment of a simple light source/detector combination.

FIG. 17 is a schematic representation of an example embodiment of a dual light path source detector combination.

FIG. 18 is a schematic representation of an example system for core body and skin temperature measurements with one spectral channel for core body temp, and the other channel comprising a thermistor for skin measurement.

FIG. 19 is a schematic representation of a wearable band such as a smart watch, a fitness indicator, or a chest strap incorporating multiple emitter/detector pairs.

### DETAILED DESCRIPTION

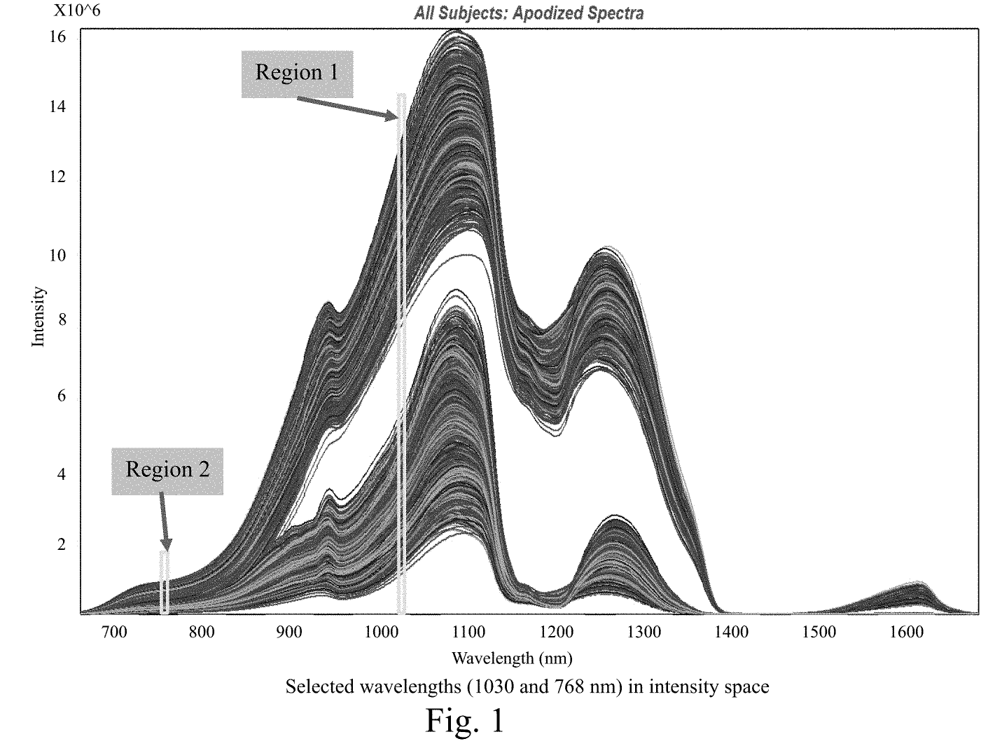

The present invention provides methods and apparatuses that can obtain depth resolved temperature in biological tissue or humans using a non-invasive interrogation of the tissue in combination with multivariate analysis or other algorithmic means. Specifically, the interrogation of tissue can be carried out by exposing the tissue with a near infrared light spectrum chosen to penetrate the skin to the desired depth, along with a detection system that captures the light and resolves the absorbance characteristics at various frequencies. FIG. 1 presents experimental data from such a system whose results are shown to correlate well with temperatures obtained from a reference ingested temperature sensor.

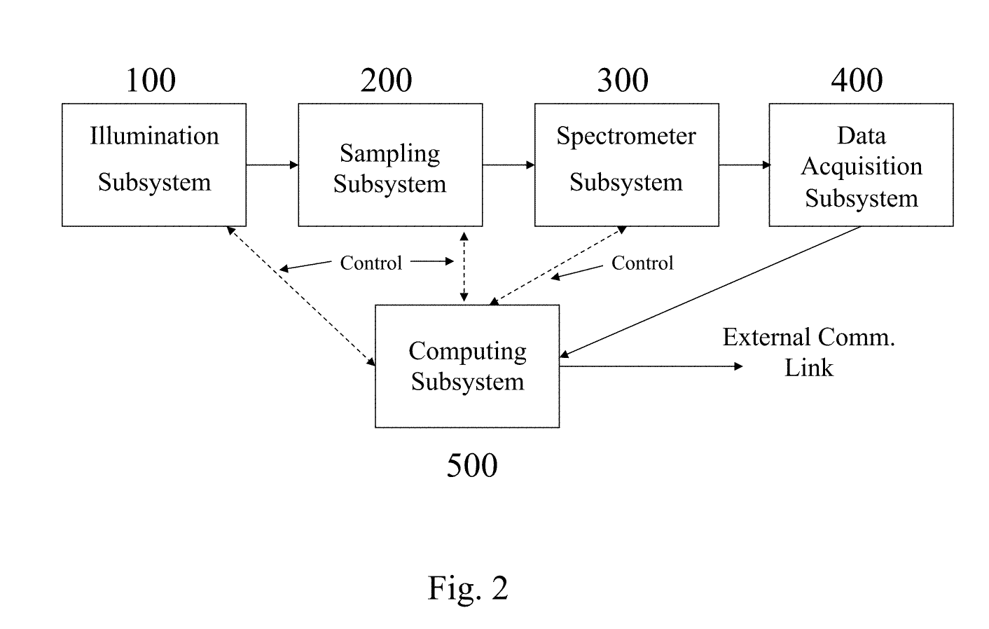

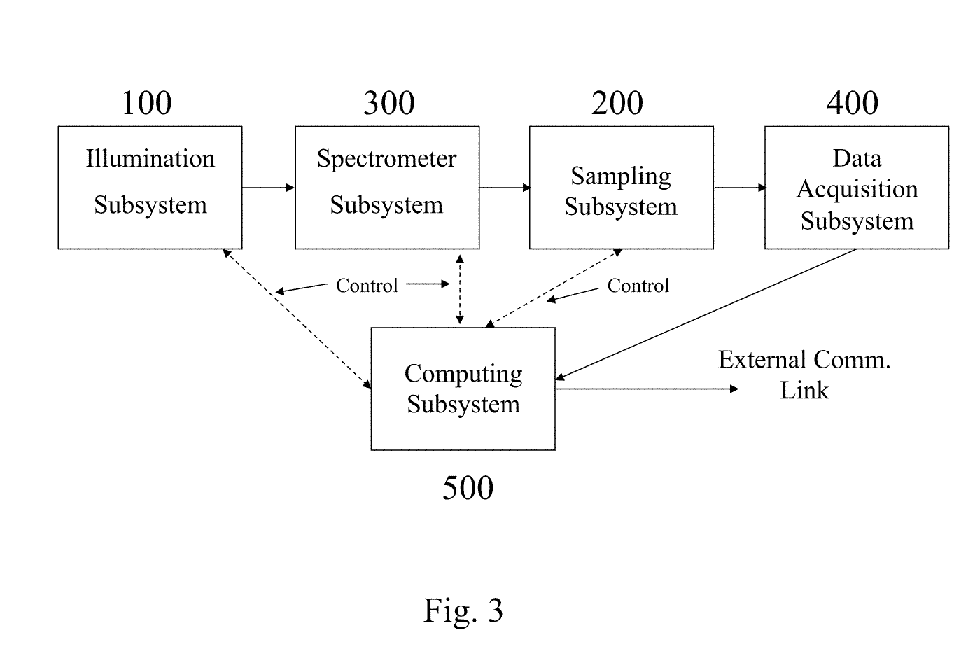

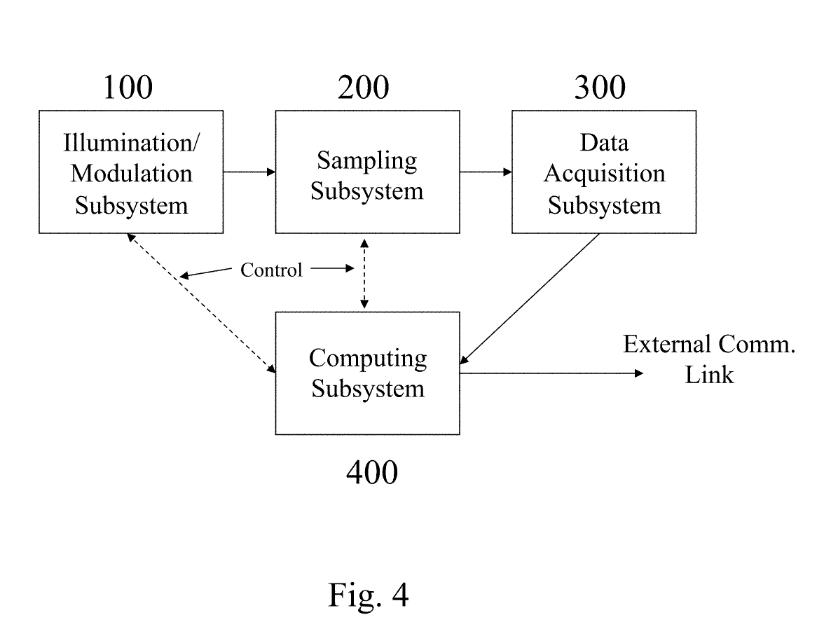

Multiple systems have been described in the referenced patents and applications referenced previously that overcome the challenges posed by the spectral characteristics of biological samples by incorporating a design that includes, in some embodiments, six optimized subsystems. The design contends with the complexities of the tissue spectrum, high signal-to-noise ratio and photometric accuracy requirements, tissue sampling errors, calibration maintenance problems, calibration transfer problems plus a host of other issues. The six subsystems, shown in FIGS. 2 and 3, include an illumination subsystem, a tissue sampling subsystem, a spectrometer subsystem, a data acquisition subsystem, a computing subsystem, and a calibration subsystem. For the purpose of discussion, the target system in FIG. 2 or 3 can be generalized by combining subsystem 200 and 300, sampling and spectrometer, into a single sampling subsystem as shown in FIG. 4.

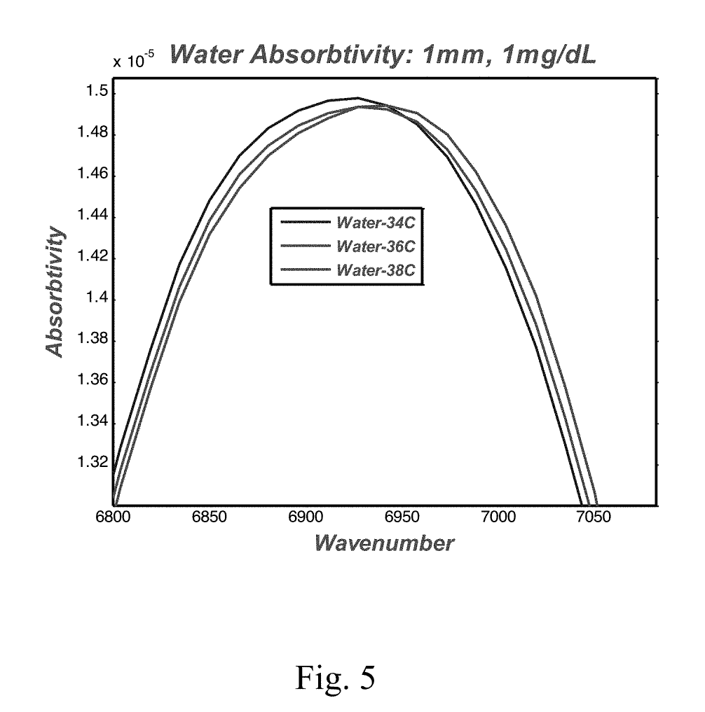

The present invention takes advantage of a property of water whereby the light absorbance characteristics of pure water change with temperature over a specified frequency range as depicted in FIG. 5. This characteristic is well mapped and understood and characterized, with some frequencies showing much larger absorbance excursions with temperature than other frequencies. Comparing those frequencies and absorbances that are subject to large temperature driven changes to other frequencies where the absorbance is minimally affected by temperature is the basis for example embodiments of the invention. The specific methods employed to overcome the difficulties inherent in tissue spectroscopy and to measure at various depths are described further herein.

In an example embodiment, the temperature measurement is made using a full spectrum of absorbance measurements (e.g., with a handheld medical spectrometer system). In this case, the wavelengths of light available might not be specifically optimized for temperature measurement but can still be suitable for use by combining multiple wavelengths. For this implementation, a linear regression model is determined wherein

T(core)=bX+Coffset

where X is the n wavelength measurement vector (in log-absorbance space, typically) and b is the empirically derived calibration regression coefficients. One skilled in the art recognizes that multiple algorithms exist for deriving b via least squared projections or other optimization functions including PLS, PCR, MLR. Additionally, traditional outlier detection and spectral correction methods can be applied to the multi-wavelength measurements.

For the purpose of this disclosure the illumination system 100 can comprise, as examples, a solid state or semi-conductor light source or a black body light source. The terms “solid state light source” or “semiconductor light source” refer to all sources of light, whether spectrally narrow (e.g. a laser) or broad (e.g. an LED) that are based upon semiconductors which include, but are not limited to, light emitting diodes (LED's), vertical cavity surface emitting lasers (VCSEL's), horizontal cavity surface emitting lasers (HCSEL's), quantum cascade lasers, quantum dot lasers, diode lasers, or other semiconductor diodes or lasers. Furthermore, plasma light sources and organic LED's, while not strictly based on semiconductors, are also contemplated in the embodiments of the present teachings and are thus included under the solid state light source and semiconductor light source definitions for the purposes of this disclosure. The term “black body light source” refers to any light source that emits radiation based upon Planck's Law or an approximation of Planck's Law. Some examples of black body light sources are filament lamps, glow bars, ceramic light sources, and passive radiators.

For the purposes of these teachings the sampling subsystem 200 indicates a spectrometer based upon any device, component, or group of components that spatially separate one or more wavelengths of light from other wavelengths. Examples include, but are not limited to, spectrometers that use one or more diffraction gratings, prisms, holographic gratings. For the purposes of these teachings the term “interferometric/modulating spectrometer” indicates a class of spectrometers based upon the optical modulation of different wavelengths of light to different frequencies in time or selectively transmits or reflects certain wavelengths of light based upon the properties of light interference. Examples include, but are not limited to, Hadamard transform spectrometers, Fourier transform interferometers, Sagnac interferometers, mock interferometers, Michelson interferometers, one or more etalons, or acousto-optical tunable filters (AOTF's). In addition, newer implementations of spectrometers based on silicon or InGaAs detector strips with corresponding absorbance filters would also apply. One skilled in the art recognizes that spectrometers based on combinations of dispersive and interferometric/modulating properties, such as those based on lamellar gratings, are also contemplated with respect to the present teachings.

The teachings make use of “signals,” described in some of the examples as absorbance or other spectroscopic measurements. Signals can comprise any measurement obtained representative of the spectroscopic measurement of a sample or change in a sample, e.g., absorbance, reflectance, intensity of light returned, fluorescence, transmission, Raman spectra, or various combinations of measurements, at one or more wavelengths. Some embodiments make use of one or more models, where such a model can be anything that relates a signal to the desired property. Some examples of models include those derived from multivariate analysis methods, such as partial least squares regression (PLS), linear regression, multiple linear regression (MLR), classical least squares regression (CLS), neural networks, discriminant analysis, principal components analysis (PCA), principal components regression (PCR), discriminant analysis, neural networks, cluster analysis, and K-nearest neighbors. Single or multi-wavelength models based on the Beer-Lambert law are special cases of classical least squares and are thus included in the term multivariate analysis for the purposes of the present teachings.

In an example embodiment, the number of wavelengths required to make an accurate measurement can be reduced by choosing the most optimal frequencies based on sensitivity or insensitivity to temperature.

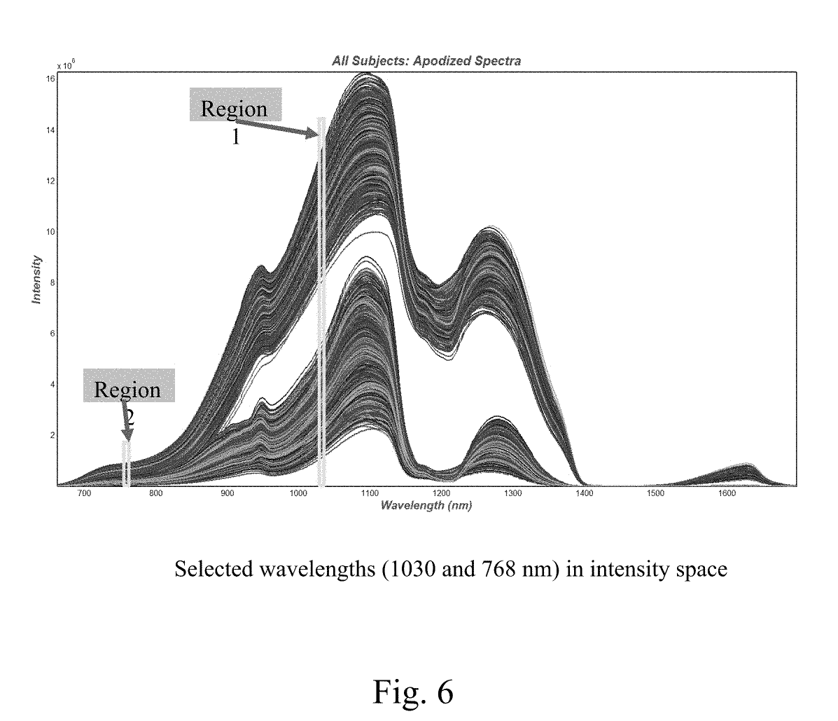

In this example embodiment, one to two wavelength sources are chosen for their correlation to temperature as shown in FIG. 6. One source, in the region of 1,000 nm to 1,100 nm and one source in the 700 nm to 800 nm region are used to produce a simple two variable measurement. For this system, a linear mathematical relationship is derived such that:

T(core)=m*log 10(S1)+b*log 10(S2)+Coffset

S1 and S2 are the intensity measurements made at wavelengths 1 and 2. m and b are instrument-dependent linear calibration coefficients that are empirically determined for the specific instrumentation implementation and temperature scale (Celsius, Fahrenheit, etc.). Coffset is a constant offset correction applied to the linear equation.

Many such frequency pairs exist and may be substituted in example embodiments.

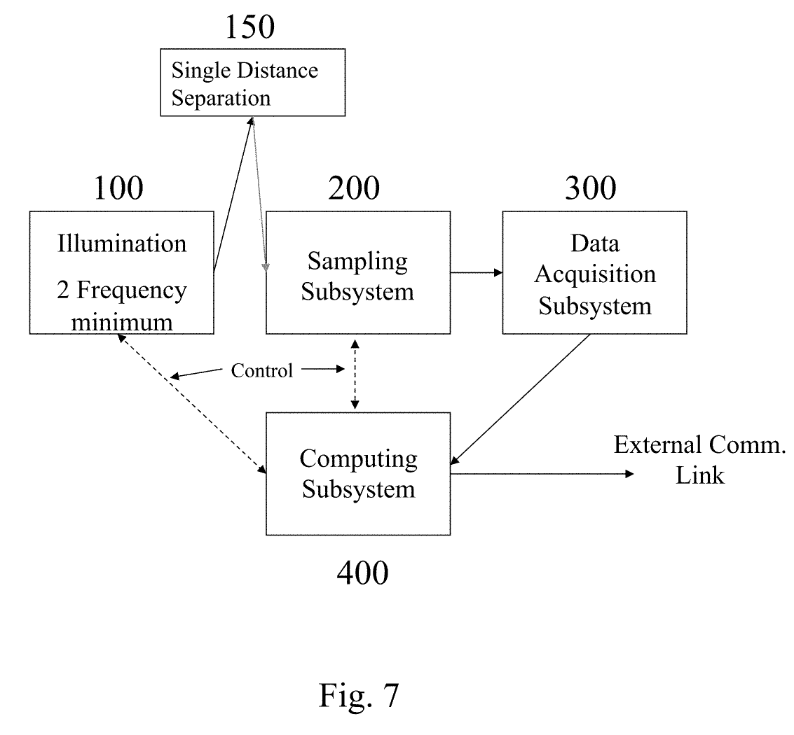

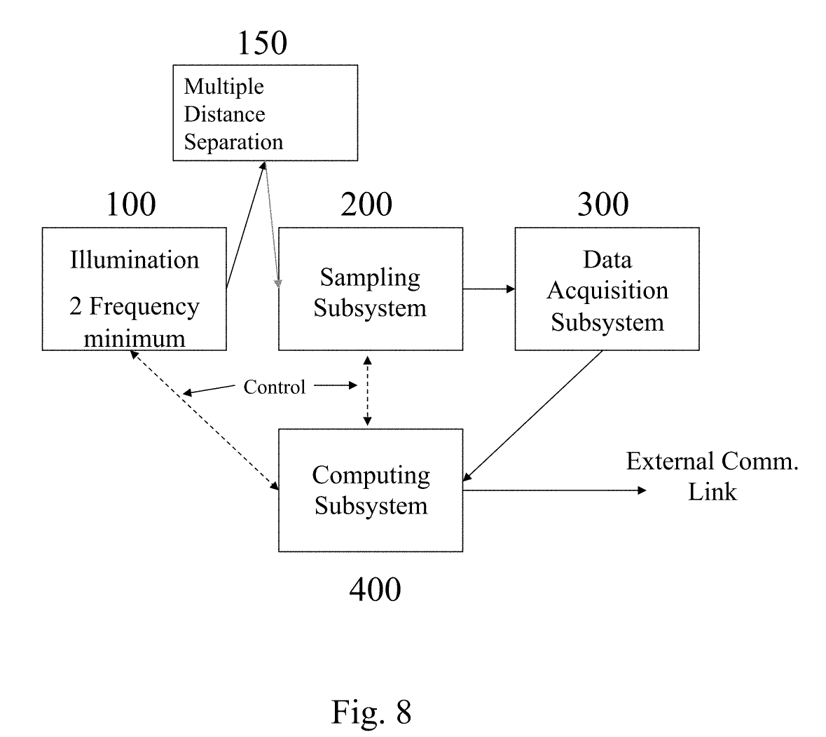

For the purpose of this reduced frequency set, the general spectrographic solution of FIG. 4 is replaced by FIG. 7 in order to measure a temperature at a single depth. The system of FIG. 8 is a general depiction of a two depth system. In this system it is assumed that one depth will be chosen to correlate generally with body core temperature, and another depth chosen to generally correlate with skin temperature, but other combinations are possible depending on clinical goals.

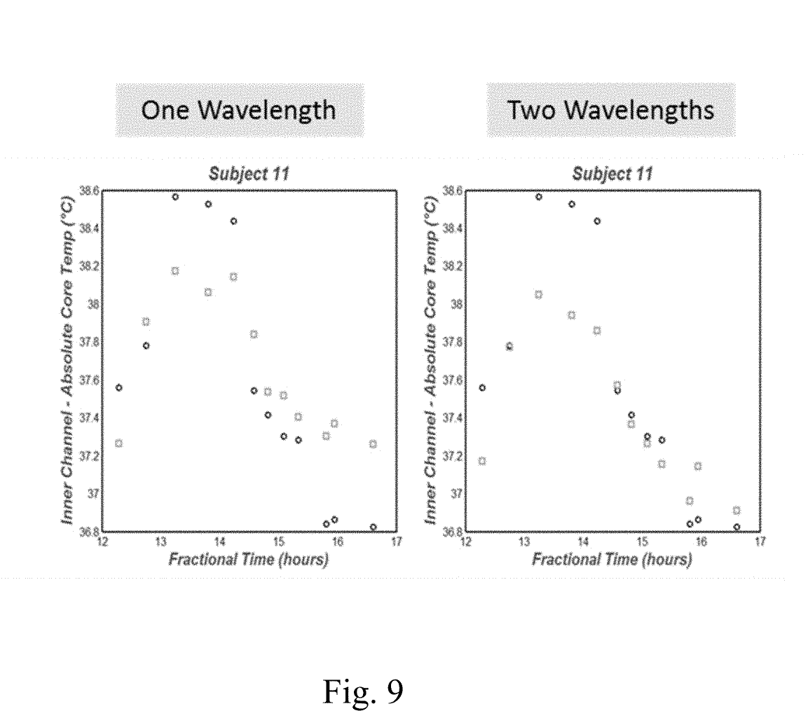

The results of an experimental single depth system using just 1 and 2 frequencies is shown in FIG. 9. The resultant fidelity is quite good with almost no optimization.

Due to the nature of body thermoregulation, a wearable or frequent episodic measurement can be assumed to follow reasonable physical limitations in terms of rate of change possible, physiological limits for living humans, etc. These physical constraints can be applied in a straightforward manner to maintain a more accurate temperature reading over time, independent of transient motion artifacts or instrumentation noise. Examples of model based algorithms include Kalman filtering and other state estimation techniques.

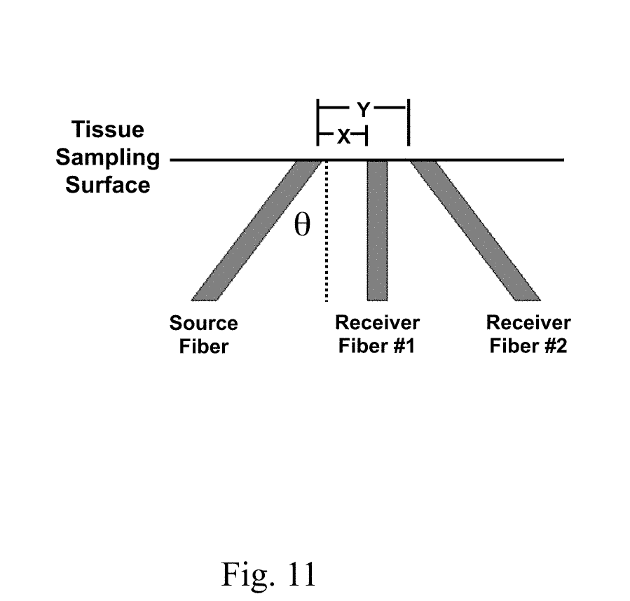

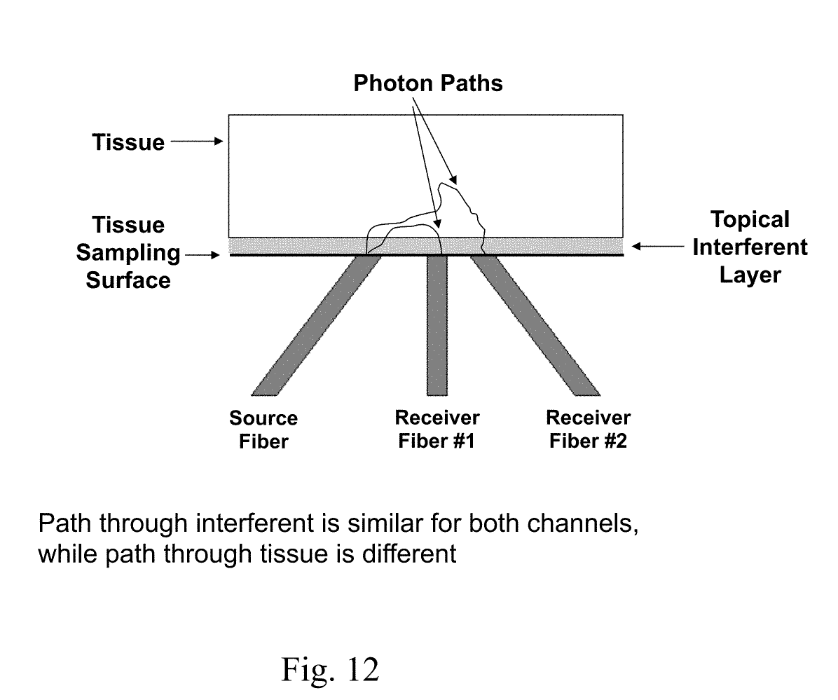

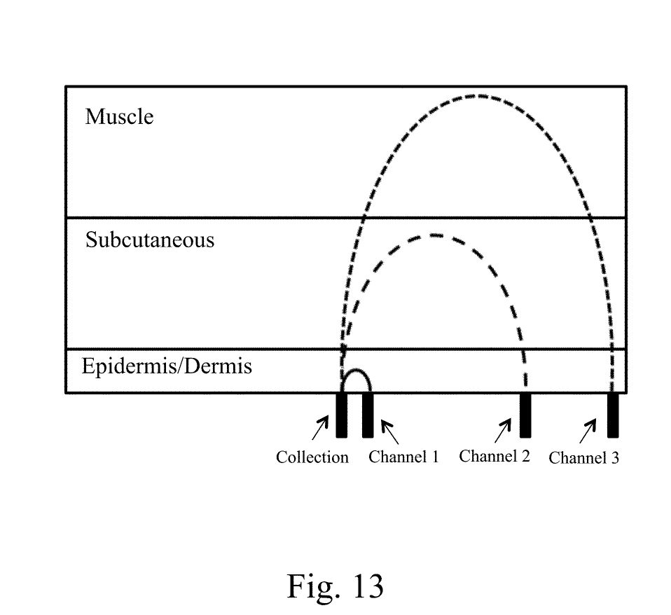

Multiple tissue depth measurements can be obtained using primarily two different techniques. Light source—receiver separation as depicted in FIG. 11 is one such method. The effective depth of the measurement will be deeper in general for wider spacing as depicted in FIGS. 12 and 13. The frequency of light is a consideration in any calculation related to depth, since some frequency ranges will be completely absorbed when attempting deeper penetrations. Wavelengths in the range of 700 to 1400 nm are generally acceptable for measurements described in this invention.

Multiple separations can be achieved by multiple receivers with a single emitter, or multiple emitters with a single receiver, or a combination of multiple receivers and multiple emitters.

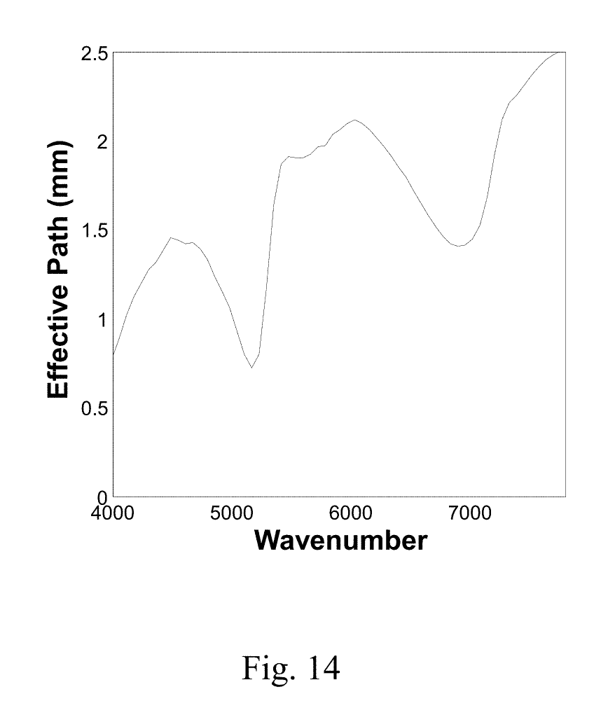

In addition to source/receiver separation, the frequency of interest also has an impact on effective depth as shown in FIG. 14.

In addition to source/receiver separation, time of flight or time frequency modulation techniques can also be used. These methods take advantage of the fact that light signals require a finite amount of time to traverse the several mm of optical path through the skin. By using fast time frequency modulation coupled with phase sensitive detection techniques, it is possible to isolate the optical data based on the total path (time) traveled in the user's body. These methods allow depth resolved data to be captured from a single source-receiver pair.

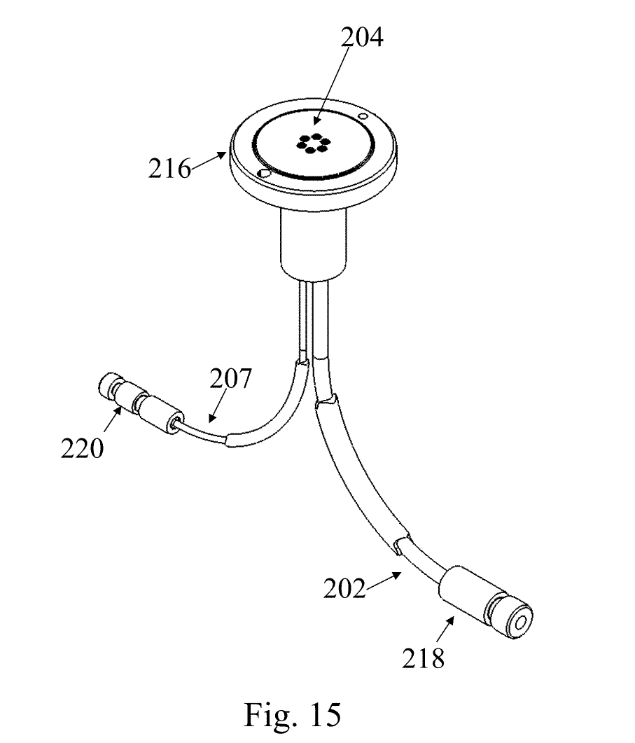

FIG. 15 is a depiction of a receiver/emitter pair suitable for use in a multiple depth continuous frequency configuration as described in FIG. 4.

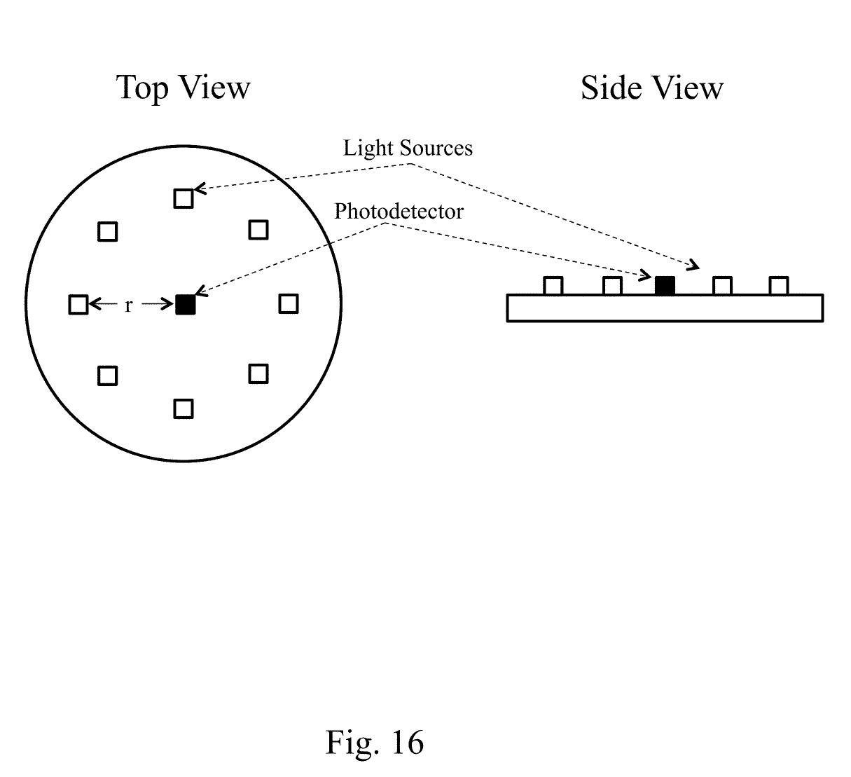

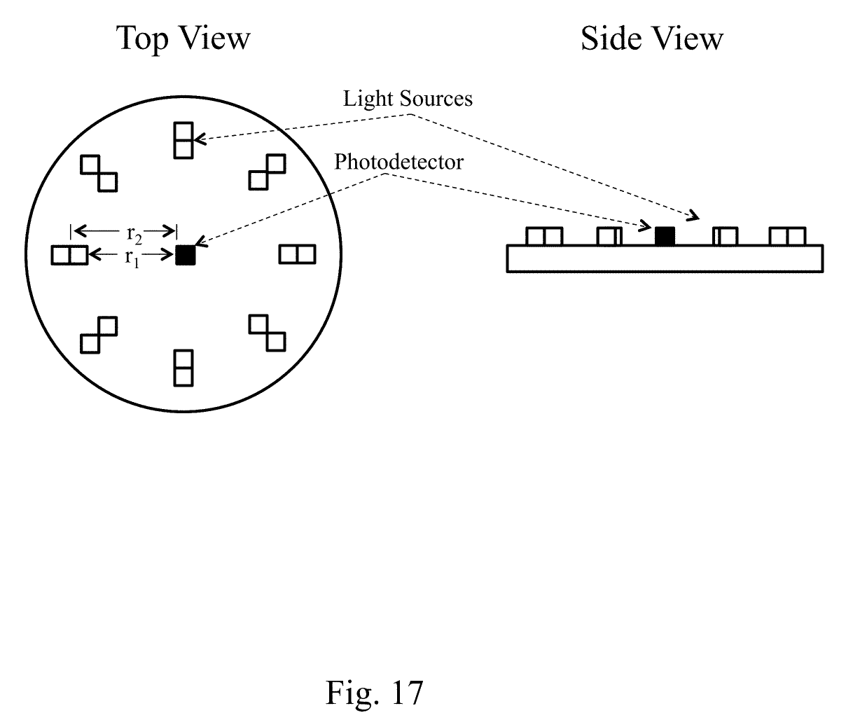

FIG. 16 and FIG. 17 show a discrete solid state emitter and receiver for single and multiple depth. This depicts a general solution for optimal illumination. The emitter ring can be replaced by single LED or laser.

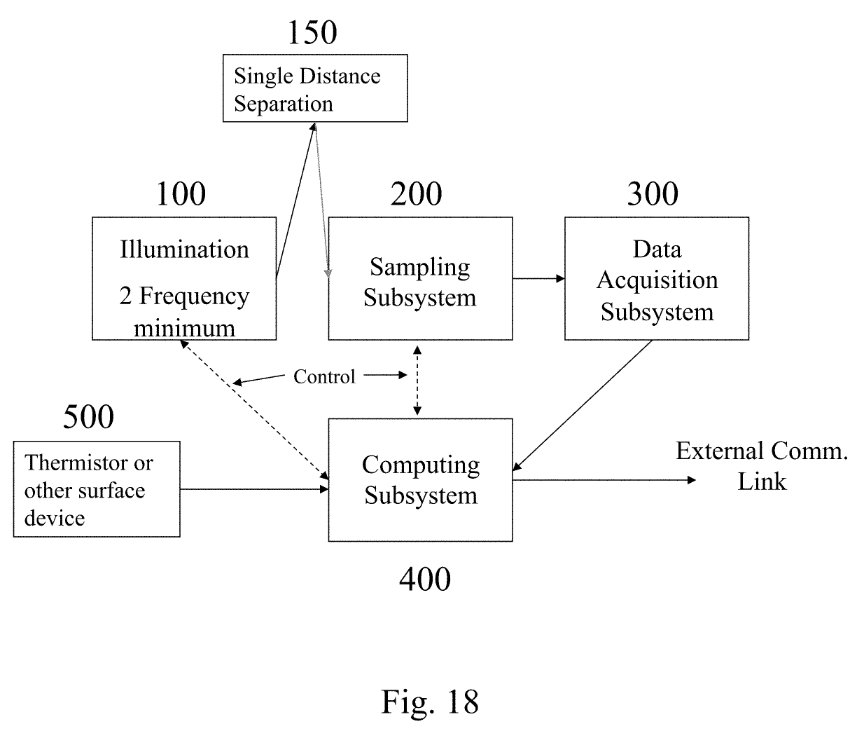

While the invention can measure temperature at various depths using multiple illumination spacing and frequencies, a differential measurement of skin to core temperature can also be achieved using spectral readings for core temperature and an alternate technology such as a thermistor, or other readily available device for skin temperature. This is depicted in FIG. 18.

A third temperature sensor can be added to measure ambient air temperature as well.

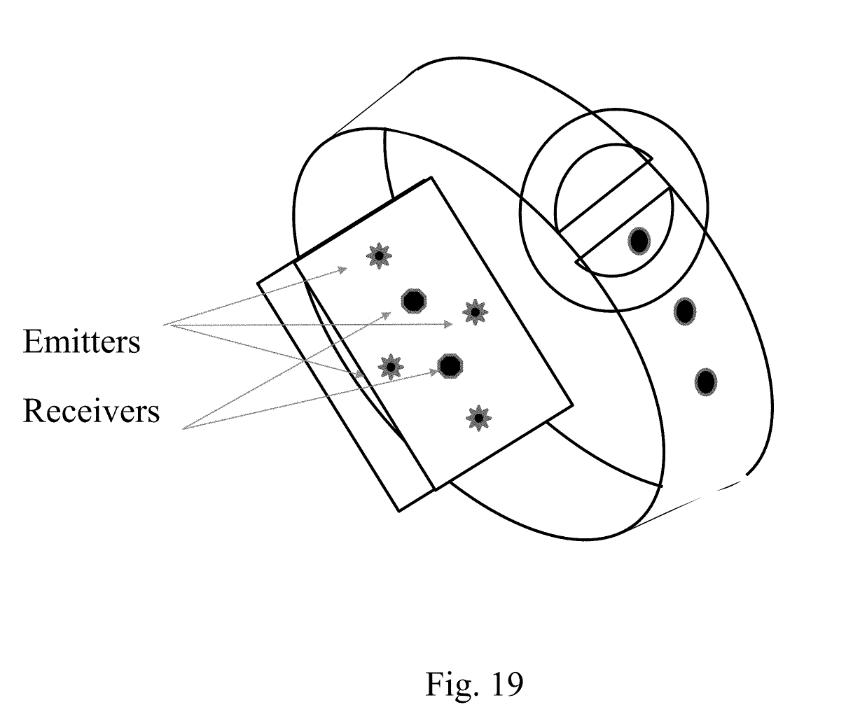

FIG. 19 depicts a general wearable solution which can comprise a smart watch, a fitness device, or a chest strap depicting multiple receiver and emitter combinations. While the figure depicts solid state emitter and receivers, as noted above, multiple combinations of solid state or black body emitters combined with receivers such as photo diodes, silicon detector strips with absorbance filters, or a grating spectrometer over the frequency of interest can also be used.

In addition, other non-wearable and wearable combinations with other sensors of interest may be combined with the core temperature sensor such as a general hydration measurement, or in combination with a pulse oximeter.

In addition to the non-imaging sensing techniques noted in the example embodiments of the present invention, there are well known multi-spectral imaging methods being used for diagnostic purposes. As the present invention is suitable for use in wavelengths of light covered by readily available commercial imaging sensors (e.g. CMOS cameras), the present invention can be used to improve the diagnostic information available for these systems. Specifically, the use of 2-dimensional patterned illumination encoding or cross-polarization filtering can provide depth resolved multi-spectral imaging information from which temperature can be determined using the present invention.

In some example applications and embodiments, the measurement can use time signals, interferometric depth resolving techniques, or both, to determine temperature profiles from specific anatomic structures. Embodiments that can provide temperature data specific to vascular structures (and hence closely correlated to core temperature) can use the pulsatile signals in a similar manner to that used in pulse oximetry. In these examples, the AC and DC light signals created by the pulseatile waveforms in the body are processed in order to separate the source of the temperature signals from the vasculature from those coming other tissues in the body.

In other example embodiments using interferometric signals, Optical Coherence Tomography (OCT) imaging methods (spectral, time domain, etc) can be used to provide depth resolved information of temperature for specific structures such as temperature profiling of organs. This can be accomplished for externally imaged structures, e.g. retinal imaging through the eye, as well for internal imaging during surgical procedures through the use of a hand-held endoscopic probe. These OCT embodiments allow absorbance spectra to be resolved at the required wavelengths for temperature measurement at specific depths thereby allowing creation of a thermal profile with depth.

Those skilled in the art will recognize that the present invention can be manifested in a variety of forms other than the specific embodiments described and contemplated herein. Accordingly, departures in form and detail can be made without departing from the scope and spirit of the present invention as described in the appended claims.

What is claimed is:

1. A temperature measurement device, comprising a spectral measurement system, configured to determine the absorbance of tissue at a plurality of frequencies, and an analysis system, configured to determine the temperature of the tissue from the determined absorbance, wherein water has a temperature dependent absorption at one of the plurality of frequencies that is different than that at another of the plurality of frequencies.

2. Methods and apparatuses as described herein.
